(19) United States
(12) Patent Application Publication
(10) Pub. No.: US 2011/0250240 A1
(43) Pub. Date: Oct. 13, 2011

(54) IMMUNE ENHANCING COMPOSITIONS AND METHODS OF USE THEREOF

(76) Inventor: Albert Crum, Rhinebeck, NY (US)

(21) Appl. No.: 13/085,957

(22) Filed: Apr. 13, 2011

Related U.S. Application Data
(63) Continuation of application No. 10/858,956, filed on Jun. 2, 2004, now abandoned.

(51) Int. Cl. A61K 33/24; A61K 9/00; A61K 38/38; A61P 31/18; A61P 25/16; A61P 25/28; A61P 9/00; A61P 3/10; A61P 17/06; A61K 31/28; A61P 35/00

(52) U.S. Cl. 424/400; 514/492; 424/617; 514/15.2

(57) ABSTRACT

A method of administering parenterally, particularly intramuscularly, glutamine and cystine and glycine plus selenium; or lactalbumin plus selenium; or lactalbumin and glutamine and cystine and glycine plus selenium, through a long-acting pharmaceutically acceptable carrier to a patient. The method comprises injecting a mixture of glutamine, cystine, glycine, lactalbumin and selenium in order to maintain the mixture systemically or locally for a sufficient time period so as to maintain blood levels of glutathione within an improved therapeutic range.

The present invention relates to formulations and compositions of amino acids and methods of parenteral administrations thereof for enhancing the immune system and methods of use of such formulations and compositions. More particularly, the present invention concerns formulations and compositions useful for replenishing vital amino acids and minerals that are depleted in individuals suffering from certain diseases wherein deficiencies of such vital amino acids are common, such as in cancer patients or patients infected with Human Immunodeficiency Virus (HIV). The formulations and compositions of the present invention provide for sustained delivery and increased uptake and absorption of amino acids and minerals to such individuals. More particularly, the present invention concerns pharmaceutical and therapeutic compositions useful for intramuscular administration of vital amino acids and minerals and nutritional supplements, which are otherwise less fully absorbed or quickly degraded. Moreover, the components of the formulations and compositions aid in the synthesis of molecules known to be free radical scavengers.

### BACKGROUND OF THE INVENTION

It is generally recognized that many disease processes are attributed to the presence of elevated levels of free radicals and reactive oxygen species (ROS) and reactive nitrogen species (RNS), such as superoxide, hydrogen peroxide, singlet oxygen, peroxynitrite , hydroxyl radicals, hypochlorous acid (and other hypohalous acids) and nitric oxide. In the eye, cataract, macular degeneration and degenerative retinal damage are attributed to ROS. Among other organs and their ROS-related diseases include: lung cancer induced by tobacco combustion products and asbestos; accelerated aging and its manifestations, including skin damage and scleroderma; atherosclerosis; ischemia and reperfusion injury, diseases of the nervous system such as Parkinson disease, Alzheimer disease, muscular dystrophy, multiple sclerosis; lung diseases including emphysema and bronchopulmonary dysphasia; iron overload diseases such as hemochromatosis and thalassemia; pancreatitis; diabetes; renal diseases including autoimmune nephrotic syndrome and heavy metal-induced nephrotoxicity; and radiation injuries. Certain anti-neoplastic drugs such as adriamycin and bleomycin induce severe oxidative damage, especially to the heart, limiting the patient's exposure to the drug. Redox-active metals such as iron induce oxidative damage to tissues; industrial chemicals and ethanol, by exposure and consumption, induce an array of oxidative damage-related injuries, such as cardiovascular pathology, cardiomyopathy and liver damage. Airborne industrial and petrochemical-based pollutants, such as ozone, nitric oxide, radioactive particulates, and halogenated hydrocarbons, induce oxidative damage to the lungs, gastrointestinal tract, and other organs. Radiation poisoning from industrial sources, including leaks from nuclear reactors and exposure to nuclear weapons, are other sources of radiation and radical damage. Other routes of exposure may occur from living or working in proximity to sources of electromagnetic radiation, such as electric power plants and high-voltage power lines, x-ray machines, particle accelerators, radar antennas, radio antennas, and the like, as well as using electronic products and gadgets which emit electromagnetic radiation such as cellular telephones, and television and computer monitors.

Mammalian cells have numerous mechanisms to eliminate these damaging free radicals and reactive oxygen and nitrogen species. One such mechanism includes the glutathione system, which plays a major role in direct destruction of reactive oxygen compounds and also plays a role in the body's defense against infection. Glutathione is a tripeptide composed of glutamine, cysteine and glycine. It is known that insufficient levels of glutathione may result in the onset of numerous diseases. These include, but are not limited to, cancer, autoimmune or immune deficiency diseases, such as rheumatoid arthritis, chronic fatigue syndrome, fibromyalgia, and AIDS. Other diseases of aging appear to be associated with a drop in glutathione levels. Moreover, since there is no evidence of transport of glutathione into cells, glutathione must be produced intracellularly.

Thus, there is a need for nutritional supplements that may aid in elimination of these damaging free radicals and reactive oxygen species. One possible mechanism for achieving this may be through enhancement of glutathione levels in patients by administration of specific compositions and stable formulations as therapeutic dosages, which contain precursors and co-factors for glutathione synthesis.

### SUMMARY OF THE INVENTION

The present invention relates to formulations and compositions containing amino acids and trace metals useful for preparation of therapeutic, immune-enhancing supplements and pharmaceuticals.

In particular, the present invention provides for the use of three amino acids, including glutamine (or as may be also inferred herein wherever “glutamine” is recited, the alternative precursor glutamic acid), cystine (or as may be also inferred herein wherever “cystine” is recited, the alternative precursor N-acetyl cysteine or various cysteine precursors/affiliates), and glycine, combined in appropriate concentrations for parenterally administered, long-acting delivery especially intramuscular (“IM”) based delivery that provides for efficient, sustained uptake and absorption. The present invention described herein provides for several different pharmaceutical compositions used in the method of the invention. Such compositions may therefore comprise a therapeutically effective amount of glutamine, cystine, glycine, and selenium; or lactalbumin and selenium; or lactalbumin, glutamine, cystine, glycine and selenium, and a pharmaceutically acceptable carrier. The compounds of the invention can be formulated to include additional essentials, such as varying levels of histidine, as known in the art, for administrations to patients exhibiting uremia. The composition, if desired, can also contain minor amounts of wetting or emulsifying agents, or pH buffering agents.

Such compositions will contain a therapeutically effective amount of the mixture, with each component of the mixture preferably in purified form, together with a suitable amount of carrier so as to provide the form for proper administration to the subject. The formulation should suit the exact mode of administration.

As mentioned, the formulation or composition also includes selenium as one of the essential components. The selenium compounds employed in accordance with the present invention are water-soluble organic or inorganic compounds containing selenium in a form capable of being absorbed by the body. Preferably, the compounds may contain selenium in the form of the selenium methionine or selenite anions, either of which may be utilized in the descriptions relating to selenium set forth herein. In the practice of the present invention, the therapeutic compositions are formulated so that the selenium compounds are present to certain specific amounts and concentrations so that the selenium content is non-toxic, yet useful for the in vivo synthesis of glutathione from its three amino acid components or precursors.

In accordance with the present invention, the therapeutic compositions and formulations contain glutamine, cystine, and glycine plus selenium in concentrations that are suitable for intracellular synthesis of glutathione. These formulations and compositions are useful for restoring glutathione levels to normal in mammals having sub-optimal levels of glutathione, due to depletion of stores of glutathione through disease or related conditions wherein such normal levels are depleted. Typically, such compositions and formulations may contain an effective but non-toxic amount of amino acids, that in an exemplary embodiment might be approximately 146 molecular weight glutamine, 240 molecular weight cystine (or 120 molecular weight for N-acetyle cysteine or for cysteine precursors), and 75 molecular weight glycine, with a ratio of 1 part glutamine: 0.5 part cystine: 1 part glycine (or 1:1:1 where N-acetyl cysteine or cysteine precursors are utilized).

The compositions and formulations of the present invention may contain, in the same ratios expressed above, lactalbumin as a source of glutamine, cystine and glycine where there is no allergic response from a patient. The lactalbumin would be combined with selenium for optimal synthesis of glutathione. Thus, the present invention provides for the delivery of such a composition by intramuscular injection so as to provide a long acting release of the above components in such a way as to provide for efficient uptake and absorption on a sustained basis. Furthermore, the present invention provides for the use of lactalbumin as a natural buffer for the delivery of exogenous glutamate, cystine and glycine to the cell. When combined with selenium, the release of the three amino acids from lactalbumin in combination with the selenium provides the appropriate components for intracellular synthesis of glutathione. Thus, due to the natural buffering capacity of the lactalbumin, the glutamine, cystine and glycine are protected from rapid degradation and disposal and remain available for synthesis of glutathione when presented intracellularly with selenium as a co-factor.

A further aspect of the invention is the combination of lactalbumin with glutamine, cystine, glycine and selenium to further optimize the interaction of the necessary components for synthesis of glutathione.

Another aspect of the invention is the parenteral delivery of glutamine, cystine, glycine and selenium in a buffered system other than lactalbumin to prevent degradation of the four components prior to intracellular uptake. The buffering system envisioned is one that mimics the protective effects of lactalbumin during delivery of the three amino acids plus selenium.

Another aspect of the invention is to provide for long-acting compositions that will provide for sustained delivery of the compound. The long-acting nature of the delivery provides the inherent advantage of minimal administrations so that those who are unavailable or incapable of receiving daily or other frequent dosages through oral, buccal, and/or other transmucosal means may still avail themselves of the therapeutic benefits of glutathione by the described IM administration. Moreover, the parenteral administration can provide superior pharmocokinetics for the dissolution, absorption and distribution phases in the course of eliciting the pharmacologic effects described herein of glutathione in the bodies of humans. The compound described herein is administered intramuscularly, the end result of which is a significant increase in bioavailability over oral and/or transmucosal routes. This is because it has been found that oral and transmucosal routes tend to impede absorption and other phases given the tendency of these routes to present obstacles such as pH degradation, macrophagic uptake, and the like, all of which can hamper efficient administration of glutathione, especially on a sustained basis. Moreover, unlike oral and some transmucosal administrations, the intramuscular administration described herein provides for a longer term delivery than that limited by delivery vehicles that are curtailed because of various natural limitations, such as digestive tract length, normal patient usage of nasal or rectal passages, etc.

Further aspects of the invention include the use of the formulations and compositions of the present invention for treatment or amelioration of diseases associated with aging including, but not limited to, lung cancer induced by tobacco combustion products and asbestos; accelerated aging and its manifestations, including skin damage, psoriasis and scleroderma; atherosclerosis; ischemia and reperfusion injury, diseases of the nervous system such as Parkinson disease, Alzheimer disease, muscular dystrophy, multiple sclerosis, stroke, senile dementia; lung diseases including emphysema and bronchopulmonary dysphasia; iron overload diseases such as hemochromatosis and thalassemia; pancreatitis; diabetes; renal diseases including autoimmune nephrotic syndrome and heavy metal-induced nephrotoxicity; and radiation injuries, arthritis, asthma, autoimmune diseases, cachexia, chronic fatigue syndrome, colitis, coronary artery disease, fibromyalgia, hepatic dysfunction, viral infections, inflammatory bowel disease, lupus, macular degeneration, multiple sclerosis, neurodegenerative diseases, nutritional disorders, and vasculitis.

A yet further aspect of the invention includes the use of the formulations and compositions for normalizing T_{H}/T_{S} cell ratios in AIDS patients, and in other immunocompromised patients.

A yet further aspect of the invention includes the use of such formulations and compositions for enhancing B cell responses in AIDS patients, and in other immunocompromised patients.

A yet further aspect of the invention is the use of the formulations and compositions for normalizing natural killer cell function and T cell function in AIDS patients ,and in other immunocompromised patients.

### DETAILED DESCRIPTION OF THE INVENTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only to the appended claims.

As used in this specification and the appended claims, the singular forms “a”, “an”, and “the” include plural references unless the context clearly dictates otherwise. Thus for example, references to “a screening assay” include one or more assays; reference to “the formulation” or “the method” includes one or more formulations, methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety to disclose and describe the methods and/or materials in connection with which the publications are cited.

### DEFINITIONS

The term “Substantially Pure,” when referring to a polypeptide or amino acid, or co-factor, including but not limited to selenium, means a polypeptide, or amino acid, or co-factor such as selenium that is at least 60%, by weight, free from the proteins and naturally-occurring organic molecules with which it is naturally associated. A substantially pure composition of glutamine or cystine or glycine or selenium is at least 75%, more preferably at least 90%, and most preferably at least 99%, by weight, of glutamine, or cystine, or glycine or selenium.

The amino acids and co-factors described herein, including glutamine, cystine or glycine, or selenium can be obtained, for example, by chemical synthesis or by isolation from natural sources. Purity can be measured by any appropriate method, e.g., column chromatography, polyacrylamide gel electrophoresis, HPLC analysis, and chiral methods. Chiral purity is important and can be assayed by known methods, including chiral chromatography or optical rotation.

“Treatment” refers to the administration of medicine or compositions or formulations or the performance of medical procedures with respect to a mammal, including a human, for either prophylaxis (prevention) or to cure or ameliorate or normalize the infirmity or malady or deficiency in the instance where the patient is afflicted or deficient.

“Patient” or “Subject” means a human or non-human mammal that may benefit from the therapies described in the present application, for example, anti-oxidant therapy. The anti-oxidants may be administered to subjects already having a disease or condition whose symptoms and sequelae are attributed to oxidation of proteins, cells or tissues, or particular molecular entities or chemical compounds, and whose symptoms or sequelae may be alleviated by anti-oxidant therapy. Alternatively, the subjects may be predisposed to diseases or conditions caused by high levels of oxidation, for which therapy with an anti-oxidant may be beneficial. Accordingly, the subject may be treated therpeutically with the anti-oxidant therapy. Diseases or conditions for which such anti-oxidant therapy would be beneficial may be selected from the group consisting of autoimmune or degenerative diseases including acetaminophen poisoning, ADD, Addison's disease, aging, AIDS, alopecia areata, ALS, Alzheimer's disease, anemia (hemolytic), ankylosing spondylitis, arteriosclerosis, arthritis (including osteoarthritis and rheumatoid arthritis), asthma, autism, autoimmune disease, Behcet's disease, burns, cachexia, cancer, candida infection, cardiomyopathy (idiopathic), chronic fatigue syndrome, colitis, coronary artery disease, cystic fibrosis, diabetes, Crohn's disease, eczema, emphysema, Epstein Barr Viral (EBV) syndrome, fibromyalgia, free radical overload, Goodpasture syndrome, Grave's disease, hepatic dysfunction (liver disease), hepatitis B, hepatitis C, HIV or patients suffering from AIDS, hypercholesteremia (high blood cholesterol), herpes, infections (viral, bacterial and fungal), inflammatory bowel disease (IBD), lupus, macular degeneration, malnutrition, Meniere's disease, multiple sclerosis, myasthenia gravis, neurodegenerative diseases, nutritional disorers, Parkinson's disease, Pemphigus vulgaris, Primary Billiary Cirrhosis, progeria, psoriasis, rheumatic fever, sarcoidosis, scleroderma, shingles, stroke, surgery, toxic poisoning, trauma, vasculitis, vitiligo, and Wegener's granulomatosis.

A “Therapeutically Effective Amount” or a “Nutritionally Effective Amount” is an amount of an agent, composition or formulation sufficient to achieve the desired treatment effect.

“Muscular” refers to any of the principle muscle groups within the human body, but most typically will refer to the gluteal and/or deltoid groups for injection purposes.

“Intramuscular delivery” and “IM” refer to administration of drugs or other agents through the epidermal layers, into one or more muscular regions of the human body.

“Parenteral” refers to the route of materials across or substantially through the epidermal layers of the human body usually by means of intravenous (IV), intramuscular (IM), or subcutaneous (SC) means.

“Pharmaceutically Acceptable” means approved by a regulatory agency of the Federal or a state government or listed in the U.S. Pharmacopeia or other generally recognized pharmacopeia for use in animals, and more particularly in humans.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

“Depot” refers to a drug delivery liquid following administration to a warm-blooded animal which has formed a gel upon the temperature being raised to or above the gelation temperature.

“Gel” means the semi-solid phase that spontaneously occurs as the temperature of the “polymer solution” or drug delivery liquid is raised to or above the gelation temperature of the block copolymer.

“Gelation Temperature” means the temperature at which the biodegradable block copolymer undergoes reverse thermal gelation, i.e., the temperature below which the block copolymer is soluble in water and above which the block copolymer undergoes phase transition to increase the viscosity or to form a semi-solid gel.

“Aqueous Polymer Composition” means either a drug delivery liquid or gel comprised of the water phase having uniformly contained therein a drug and a biodegradable block copolymer. At temperatures below the gelation temperature, the copolymer may be soluble in the water phase and the composition will be a solution. At temperatures at or above the gelation temperature copolymer will solidify to form a gel with the water phase, and the composition will be a gel or semi-solid.

“Poly(lactide-co-glycolide)” or “PLGA” shall mean a copolymer derived from the condensation copolymerization of lactic acid and glycolic acid, or, by the ring opening polymerization of a-hydroxy acid precursors, such as lactide or glycolide. The terms “lactide”, “glycolide”, and “glycolate” are used interchangeably.

### GENERAL ASPECTS OF THE INVENTION

Traditional administrations of antioxidants are usually administered through oral or transmucosal means, and are intended to bring the levels of amino acids, vitamins, minerals and other materials needed for proper nutrition to the desired values. They may serve as a source of essential amino acids which may or may not be present in foods, but in many cases may ultimately be destroyed or simply may not synthesized by metabolic processes in a mammal suffering from certain diseases or whose metabolism is impaired while undergoing certain types of therapies.

Generally, the efficiency of absorption of orally administered agents such as polypeptides is very poor, due to a number of factors, including various proteases in the gastrointestinal tract that metabolize polypeptides or by way of a myriad of hepatic metabolic events which further degrade such agents. As a result of the poor absorptive efficiency of orally administered therapeutic agents such as polypeptides, it is necessary to administer large doses of such agents. This is costly and inefficient.

Moreover, in certain conditions, including, but not limited to, neurological conditions, the ability to swallow is impaired, thus leading to the necessity for delivery of nutrients via feeding tubes, or through intravenous administration. However, even this method may lead to less than desirable results because of the constant need for administration, given the immediate, rather than sustained level of bioavailability resulting from the absence of an absorption phase in venous circulation methods.

Thus, there is a need for a minimally invasive, yet highly efficient and absorptive method of sustained delivery to patients of therapeutic polypeptides or amino acids, which may be degraded when administered orally or otherwise not available for long term delivery.

Moreover, it is known that an important antioxidant like glutathione, in addition to poor oral absorption is eliminated by the liver through the bile without ever reaching the bloodstream when administered in other ways, like in the various transmucosal methods known in the art. Thus, oral, intravenous administration, and various transmucousal methods known in the art all harbor severe limitations as vehicles of glutathione administration, and in particular, do not have a sustained or long-acting effect in replenishing glutathione in disease states where glutathione levels are depleted.

An alternative approach for boosting intracellular levels of glutathione is to utilize intramuscular administration of the precursors for glutathione along with specific co-factors necessary for its synthesis in a stable composition and formulation, such that these precursors and co-factors are available for the synthesis of glutathione before any endogenous breakdown can occur.

Though the influx of the three precursor amino acids needed for glutathione synthesis, including cysteine, glutamine and glycine, into cells is limiting, cysteine appears to be the rate limiting step in glutathione synthesis. However, free cysteine is toxic and spontaneously oxidized. Thus, a more reasonable approach would be to use cystine, which upon reduction of the disulfide bonds intracellularly to form cysteine, would be a safe and effective means of achieving the desired result.

Thus, although the role of glutathione is important for detoxification of chemical pollutants, carcinogens and ultraviolet light radiation by virtue of its ability to eliminate oxyradicals, or to eliminate free radicals produced normally during cellular metabolism, the problem still exists as to how to replenish the levels that are deficient in various diseases associated with aging, as well as in non-age related conditions.

Thus, there is a need for development of novel compositions and formulations for restoration and normalization of intracellular glutathione levels as a means for detoxification of these damaging radicals, particularly in a way that avoids any of the above detailed shortcomings of oral or transmucosal adminstrations.

In view of the role that glutathione plays in protection of cells from the damaging effects of free radicals, and the need for replenishing intracellular supplies of glutathione in certain disease states where deficiencies exist, intramuscular delivery systems are described herein for transport of the precursor amino acids and co-factors needed for de novo synthesis of glutathione. The results shown below in the Example section demonstrate how the compositions and formulations of the present invention may be used to treat individuals suffering from certain disease states or have nutritional deficiencies where restoration of glutathione levels is desired.

### Pharmaceutical Compositions and Methods of Administration

The compositions and formulations of the present invention are suited for the intramuscular delivery of the precursor amino acids and co-factors for synthesis of glutathione. These amino acids are glutamine, cystine and glycine. Although the three amino acid constituents of glutathione are glutamine, cysteine and glycine, cysteine is known to be toxic, and thus cannot be used in the context described herein. Thus, the compositions and formulations of the present invention incorporate the use of cystine, which, upon reduction by intracellular reductases, will form cysteine. When these three amino acids are combined with the fourth component of the composition, selenium, a cofactor for glutathione synthesis, glutathione levels are enhanced. Likewise, other compositions of the present invention comprise lactalbumin plus selenium or lactalbumin plus glutamine, cystine, glycine and selenium.

The compositions and formulations of the present invention are suited for intramuscular administration of lactalbumin, glutamine, cystine, glycine and selenium, preferably via an injectable, biodegradeable sustained release delivery systems for the purpose of maintain the mixture systemically or locally for a sufficient time period so as to maintain blood levels of glutathione within an improved, if not optimal, therapeutic range for a given patient. As such, the compositions of the instant invention comprise glutamine, cystine, glycine and selenium; or lactalbumin plus selenium; or lactalbumin, glutamine, cystine, glycine plus selenium dissolved or dispersed in a sustained release carrier.

Various sustained release intramuscular dosage forms such as suspensions, oil depots, liposomes, and implants may be envisioned for carrying or delivering the above described compositions, in a preferred embodiment, the carrier will be a polymer based vehicle, such as polymeric microspheres and/or gels/gel depots. Exemplary delivery methodologies for other compositions are known in the art, and may be further detailed in publications such as U.S. Pat. Nos. 6,331,311; 6,461,631; 5,945,115; 6,011,011; 6,117,949; 5,019,400; 6,706,289; 6,592,908 and 4,938,763, as described in “Remington's Pharmaceutical Sciences” by E. W. Martin, and “The Handbook of Pharmaceutical Excipients, published by The American Pharmaceutical Association and The Pharmaceutical Society of Great Britain” (1986), and “The Handbook of Water-Soluble Gums and Resins”, ed. By R. L. Davidson, McGraw-Hill Book Co., New York, N.Y. (1980), all of which are hereby incorporated by reference, may be utilized as a basis for customizing an intramuscular, long-acting delivery of the composition detailed above.

By way of illustration only, one such preferred embodiment comprises a biodegradeable, bioacceptable polymer solubilized in a solvent. In an especially preferred embodiment, this polymer would be a lactic acid-based polymer, ideally PLGA (polylactide-co-glycolide), having a monomer ratio of lactic acid to glycolic acid in the range of 100:0 to about 15:85 (most preferably 75:25), and a number average weight from 1,000 to 120,000.

The polymer would be solubilized in a solvent, like a polar organic liquid, such as those from the group comprising methylene chloride, acetone, chloroform, ethyl acetate, etc. Alternatively, the solvent may be a non-toxic alcohol known in the art as being useful in such formulations of the present invention and may include, but not be limited to, ethanol, isopropanol, stearyl alcohol, propylene glycol, polyethylene glycol, and other solvents having similar dissolution characteristics.

The composition described above may be micronized as known in the art of pharmaceutical micronization for addition to the above carrier. By way of an illustrative micronization process, the composition could be solubilized in a suitable solvent (such as NaHCO₃ buffer dissolved in either deionized water or sterilized water for injection), and then stabilized, if necessary, by complexing it with say, a metal cation to avoid aggregation, and to ensure a proper pH that is commensurate with the localized injection site (in this case, muscular tissue, which has a pH around 6.0). The complexed composition can be micronized using an ultrasonic nozzle as known in the art, so as to spray the same into liquid nitrogen in order to micronize the particles to a preferred diameter of 1-6 microns. Lyophilization according to this method will form stable glutathione precursor composition particles for addition to the above described polymer solution, so that thereafter the polymer solution with stabilized composition particles may be processed to form microspheres by creating droplets from an ultrasonic or pressure nozzle and placing the same onto or near liquid nitrogen on top of a frozen nitrogen bed, where they are then removed and exposed to a non-solvent (e.g., ethanol or ethanol mixed with hexane or pentane) to extract the polymer solvent (methylene chloride, chloroform, etc.) The end result will be an active (the “composition”, e.g., the above described glutathione precursors and applicable essentials) encapsulated within the excipient or delivery vehicle, together forming a freely flowing micronized powder that may be reconstituted with sterilized water prior to injection. Of course, many other forms of processing known in the art for forming injectable PLGA microspheres containing actives are explicitly contemplated in the present application.

Thus, the injectable form is delivered into muscle tissue by microspheres that encapsulate the drug within a biodegradable polymer. Thereafter, the microspheres undergo gradual hydrolysis, resulting in a gradual release of above described components or precursors into the patient system. In some cases, full release of the long-acting composition from the gradually hydrolyzing microspheres may have a delayed start after an intramuscular injection. Accordingly, supplemental buccal or other transmucosal glutathione precursors may be recommended during the first IM injection(s). Release is targeted to be sufficient to maintain blood levels of 200-400 moles/L of glutathione during the administration sustained release period (ranging from 1-4 weeks/administration), after which the composition is absorbed completely from the microspheres, which are biodegraded to carbon dioxide and water through the Krebs cycle.

The invention provides methods of treatment comprising administering to a subject an effective amount of glutamine, cystine, glycine, and selenium; or lactalbumin and selenium; or lactalbumin, glutamine, cystine, glycine and selenium. In any of these embodiments, the compound may be substantially purified (e.g., substantially free from substances that limit its effect or produce undesired side-effects). In the above described lactalbumin embodiments, the compound may be formed as generally described above, but may further be formulated so as to result in an administration that will have approximately 0.5 grams of lactalbumin as a baseline means of achieving the desired serum and bloodstream levels described herein, but will of course depend on the particular subject and condition ascribed thereto. The subject is preferably a human although the compositions and formulations of the present invention may be administered to animals, including but not limited to dogs, cats, cows, pigs, horses, chickens, etc. In one specific embodiment, a non-human mammal is the subject. In another specific embodiment, a human mammal is the subject.

Nevertheless, as can be further appreciated by those skilled in the art, the amount of each of the components in the present invention that is optimal in protecting against free radical damage can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances.

The intramuscular administration of the components of the present invention, in an illustrative preferred embodiment can be accomplished generally by injecting between up to 2 -5 ml (generally up to 2 ml to the deltoid muscle group, or up to 5 ml to the gluteal muscle group) over a sufficient time period to induce the desired therapeutic effect. Of course, the precise amount of the composition or formulation administered according to the present invention is dependent on numerous factors, such as age and body weight of the patient or animal, the condition of the patient or animal and the desired duration of use. The time period, as described above, can be in the range of 1 to 4 weeks in duration, depending on the medical need, and according to the ratio of lactide to glycolide as also described above, and as known in the art for preparing varying long-acting polymer structures.

The administration of the compounds of the present invention can be alone, or in combination with other compounds effective at treating the various medical conditions contemplated by the present invention. Also, the compositions and formulations of the present invention, may be administered with a variety of analgesics, anesthetics, or anxiolytics to increase patient comfort during treatment. Appropriate doses for any particular host can be readily determined by empirical techniques that are well known to those of ordinary skill in the art.

### EXAMPLES

### Example 1

### Treatment of Infectious Diseases

The production and release of free radicals and other reactive oxygen species occurs following the immunological response to infection by a pathogenic organism. The production of such oxyradicals occurs as a protective means of eliminating the invading organism, but it also poses a risk for the host at the same time, since the free radicals produced not only eliminates the pathogen, but also has a deleterious effect on the host cell. This is especially true of chronic infections, whereby the host is constantly exposed to the presence to such damaging effects of free radicals. Accordingly, administration of the present invention's compositions and formulations are contemplated as efficacious for the treatment of various infectious diseases, these including infections caused by microorganisms including bacteria, viruses, and protozoal infections. The compounds of the present invention are administered through the routes of administration discussed above in the doses described above, either alone or in conjunction with an antimicrobial compound.

### Example 2

### Autoimmune/Inflammatory Disorders

Many autoimmune disorders share a common feature in that the presence of an over-reactive inflammatory response is a contributing factor to the pathology of the disease. One common feature to these diseases is the release of free oxygen radicals and reactive oxygen species by inflammatory cells, at the site of tissue injury.

### Multiple Sclerosis

One example of this is multiple sclerosis (MS). In MS, autoreactive T-cells initially begin to attack the myelin sheath surrounding neurons. Further neuronal damage is then caused by the release of reactive oxygen species and free radicals at the site. Treatment with the compositions and formulations of the present invention can aid in protection by the reactive oxygen species and free radicals. They can be administered alone or in conjunction with other compounds known to be effective in treating or controlling the MS disease state. Administration is started at the onset of disease symptoms and continued throughout the course of the disease.

### Rheumatoid Arthritis

Another autoimmune disease that may benefit from treatment with compositions and formulations of the present invention is rheumatoid arthritis. This disease begins with local tissue damage in the joints that leads to further tissue damage mediated by autoreactive T-cells. This is then followed by infiltration of pro-inflammatory cells like phagocytes which increase the damage by releasing free radicals and other oxygen reactive species. Once again, patients with rheumatoid arthritis may benefit from compositions and formulations of the present invention, which may be combined with other therapeutic agents used to reduce the inflammatory response throughout the course of the disease.

### Inflammatory Bowel Disease

Inflammatory bowel disease (IBD) is a general term for a group of chronic inflammatory disorders of unknown cause involving the gastrointestinal (GI) tract. Crohn's disease and ulcerative colitis are both chronic inflammatory disorders that fall within the scope of IBD. Both diseases have pronounced inflammation in the small intestinal mucosal tissue that can extend to other layers of the organ. Phagocytic cells are the primary drivers of the inflammatory reaction. As these cells release free radicals and reactive oxygen species in response to the inflammation, the intestinal mucosa is damaged, leading to potentially serious consequences for the patient, including sepsis. Thus, the compositions and formulations of the present invention could significantly impact on the pathogenesis of these diseases. Once again, other anti-inflammatory compounds may be used in concert with the compositions and formulations of the present invention.

The compositions and formulations of the present invention should be administered as soon as the diagnosis of IBD is made. Administration is continued during and after the hallmarks of the IBD state are detected.

### Example 3

### Neurodegenerative Diseases

Free radicals and reactive oxygen species can be relevant to a number of other neurodegenerative diseases such as ALS, Parkinson's disease, stroke and Alzheimer's. The production and release of free radicals and reactive oxygen species can cause or exacerbate the destruction of neurons in these diseases. Accordingly, the administration of the compositions and formulations of the present invention may have a positive outcome on preventing the cellular damage in these various neurological disease processes. The treatment should be initiated as soon as the initial diagnosis is made. Treatment should continue as long as symptoms persist. Other treatments for these various conditions may be administered concurrently, if available.

### Example 4

### Other Disease States

### Radiation Injury

Ionizing radiation is a harmful form of energy that damages tissue through the action of charged particles. Damage can result to tissues exposed to ionizing radiation through the effect of the energy on water, oxygen, and other molecules with the formation of reactive oxygen species and free radicals, such as free hydroxyl radicals and other highly reactive oxygen species. Moreover, tissue damage and destabilization of homeostatic equilibrium due to overexposure to radiation can result in a systemic respiratory burst from inflammatory cells such as macrophages and neutrophils. This burst results in a release of free radicals and reactive oxygen species causing tissue damage. A patient exposed to toxic levels of ionizing radiation may be treated by administering the compositions and formulations of the present invention at the time of treatment with an appropriate therapy or a diagnosis of radiation toxicity. The above procedure is repeated until an objective regression of symptoms is observed. The therapy is continued even after a partial response has been observed. In patients with complete responses, the frequency of therapy is reduced.

As Adjunct Therapy with Cancer Chemotherapy

It is known that treatment with various chemotherapeutic agents results in damage to not only cancer cells but to normal cells as well. An inflammatory response may occur following treatment with chemotherapeutic agents as a mechanism to rid the body of dead cellular debris. Thus, treatment with the compositions and formulations of the present invention may aid in prevention of further cellular damage resulting from the free radicals and reactive oxygen species released by the inflammatory cells at the site of cellular damage. Treatment with the compositions and formulations of the present invention would be concurrent with cancer chemotherapy and would continue until chemotherapy is stopped.

### Example 5

### Synthesis, Preparation of Block Polymer Containing Glutathione Precursors

Lactide and glycolide monmers would be added (in mole ratios of say, 3:1, respectively) to a flask with the minor addition of enablers (such as 0.1% wt of stannous octoate, etc.), the heated under a vacuum (say 5 mm Hg) at temperature such as 150 degrees Celsius, and the progress of the reaction monitored by available means (chromatograph. etc.). After the appropriate time, the flask would be cooled to room temperature. The residue would be dissolved in cold water and heated to 70-80 degree Celsius to precipitate the polymer formed. The supernatant would be decanted and the polymer residue would again be dissolved in cold water and heated to induce precipitation. This process of dissolution followed by precipitation might be repeated approximately three times. Finally, the polymer would dissolved in a minimum amount of water and disaggregant added, and then lyophilized for stability purposes. The resulting PLGA copolymer might have a weight average molecular weight (Mw) of approximately 3000. The PLGA might then be solubilized in solvent like chloroform to form a polymer solution. A second disaggregant (such as a metal cation) might be added as needed.

The glutathione components or precursors would be solubilized in monomer form, within a solvent such as NaHCO₃ buffer dissolved in deionized water, and then stabilized by complexing with a metallic ion such as Zn⁺² at a pH of 6.0. The complexed components would be micronized with an ultrasonic nozzle, spraying it into liquid nitrogen, thereby freezing the particles with a diameter that should be about 4 microns, and formed as lyophilized particles to be added to the above described polymer solution.

Thereafter, the polymer solution and stabilized glutathione components dispersed therein would be processed to form microspheres. Microsphere formation would be accomplished by creating droplets that would be sprayed from an ultrasonic nozzle and immediately frozen by directing the droplets onto liquid nitrogen that would be on top of a frozen ethanol bed. Once accomplished in this manner, the microdroplets would be removed from the liquid gas and exposed to a liquid non-solvent (ethanol mixed with pentane) to extract the chloroform solvent from the polymer.

### Example 6

### In Vitro Degradation of Block Polymer Containing Glutathione Precursors

The in vitro degradation of the PLGA block copolymer of example 5 would be determined for say, a 23% by weight solution (1 ml)of the copolymer incubated at different temperatures (-10 degrees Celsius, 5 degrees Celsius, 23 degrees Celsius 37 degrees Celsius). The degradation of this block copolymer would be caused by hydrolysis and would result in lactic acid and glycolic acid as the final degradation products. Samples (50 microliters) would be taken weekly. The samples would be lyophilized, dissolved in chloroform, and the molecular weights of the polymer residues determined by chromatography. The degradation of the polymer might be substantially independent of initial pH over the 3.0 to 6.0 range which can be attributed to acidification of the media as the polymer hydrolyzed to form lactic acid and glycolic acid. The thermal gelling behavior would also independent of pH over the same pH range. The degradation would be more rapid at higher temperatures.

### Example 7

### In Vivo Testing of the Block Polymer Containing Glutathione Precursors

The in vivo biodegradation of the polymer of Example 5 would be determined over a four week period. A 0.40 to 0.45 ml sample of a cold aqueous solution containing 23% by weight of the block copolymer would be injected subcutaneously into rats. Upon reaching body temperature, and after being reconstituted as part of the injection and/or through contact with muscular fluids biodegradation of the polymer would commence. Samples would be surgically retrieved as a function of time and indicated that the quantity and/or size of microspheres would became progressively smaller over a two week period, with perhaps less than 50% of the original aqueous polymer solution remaining at the site of injection. Microscopically, any small pockets of semi-viscous liquid that might not have dispersed through the bloodstream would most likely be resorbed completely over the following two week period. Additional testing would reveal that altering the lactide/glycolide ratios will alter the length of the sustained release, and hence alter the overall dissolution profile for IM administrations. It would be seen that a 50:50 lactide/glycolide ratio would have a degradation that might last for at least 15-20 days, while the 65:35, 75:25, and 88:15 lactide/glycolide ratios might have progressively longer in vivo lifetimes that might last as long as 30 or more days.

### Example 8

### In Conclusion: Sustained Release of the Block Polymer Containing Glutathione Precursors Through IM Administration

The above description will enable one skilled in the art to make PLGA type block copolymers that form aqueous solutions having sustained release properties and to utilize the same in the field of drug delivery. Although the controlled delivery of both a glutathione precursor mixture and a glutathione mixture with adjuvants are illustrated in the examples to show the functionality of sustained release IM formulations formed from aqueous solutions of a block copolymer, these descriptions are not intended to be an exhaustive statement of all variants which can be utilized and loaded into the preferred biodegradable block copolymer, or alternative copolymers such as PLA (poly(lactide)), and many others known in the art of sustained release carriers. Certainly, numerous other variants of the therapeutic glutathione preparation are well suited for delivery from aqueous compositions of block copolymers as described in this description of the invention. Similarly, neither are all block copolymers which may be prepared, and which demonstrate the critical reverse sustained release property, specifically shown. However, it will be immediately apparent to one skilled in the art that various modifications may be made without departing from the scope of the invention which is limited only by the following claims and their functional equivalents.

1-11. (canceled)

12. A parenterally administered long acting therapeutic composition comprising:
(a) a biodegradable polymer-based pharmaceutically acceptable carrier for sustained release; and
(b) a catalytic quantity of selenium together with amounts of at least glutamine, cystine, and glycine in concentrations that are suitable for intracellular synthesis of glutathione.

13. The parenterally administered long acting therapeutic composition of claim 12, wherein said biodegradable polymer-based pharmaceutically acceptable carrier for sustained release is microsphere based.

14. The parenterally administered long acting therapeutic composition of claim 12, wherein said biodegradable polymer-based pharmaceutically acceptable carrier for sustained release is a gel depot.

15. The parenterally administered long acting therapeutic composition of claim 12, wherein said biodegradable polymer of said polymer-based pharmaceutically acceptable carrier is a lactic acid-based polymer.

16. The parenterally administered long acting therapeutic composition of claim 15, wherein said lactic acid based polymer has a monomer ratio of lactic acid to glycolic acid in the range of 100:0 to about 15:85.

17. The parenterally administered long acting therapeutic composition of claim 15, wherein said lactic acid based polymer has a monomer ratio of lactic acid to glycolic acid of 75:25.

18. The parenterally administered long acting therapeutic composition of claim 15, wherein said lactic acid based polymer has a number average weight from 1,000 to 120,000.

19. The parenterally administered long acting therapeutic composition of claim 12, wherein said therapeutic composition further comprises approximately 0.5 grams of lactalbumin in addition to said selenium per a given dosage of said composition.

20. The parenterally administered long acting therapeutic composition of claim 15, wherein the glutamine, cystine and glycine are in a molar ratio of 1:0.5:1.

21. The parenterally administered long acting therapeutic composition of claim 15, wherein the catalytic quantity of selenium together and the amounts of glutamine, cystine, and glycine are sufficient to maintain a blood concentration of 200-400 moles/L of glutathione in a mammal.

22. The parenterally administered long acting therapeutic composition of claim 15, wherein the selenium, glutamine, cystine, and glycine are lyophilized particles.

23. A method of treating an immunocompromised patient patients comprising administering an effective amount of the therapeutic composition according to claim 12.

24. The method of claim 23, wherein the immunocompromised patient is selected from the group consisting of cancer patients, AIDS patients, and patients undergoing radiotherapy or chemotherapy.

25. A method of ameliorating or preventing a disease in a patient comprising administering an effective amount of a composition according to claim 12, wherein the the disease is-selected from the group consisting of Alzheimer's disease, arteriosclerosis, arthritis, asthma, autoimmune diseases, cachexia, chronic fatigue syndrome, colitis, coronary artery disease, diabetes, stroke, senile dementia, fibromyalgia, hepatic dysfunction, viral infections, inflammatory bowel disease, lupus, macular degeneration, multiple sclerosis, neurodegenerative diseases, nutritional disorders, Parkinson's disease, psoriasis, vasculitis, and scleroderma.

26. A method of ameliorating or preventing nutritional disorder in a patient comprising administering an effective amount of a composition according to claim 12.
